(19) United States
(12) Patent Application Publication
(10) Pub. No.: US 2003/0195212 A1
(43) Pub. Date: Oct. 16, 2003

(54) Novel melanocortin receptor agonists and antagonists

(76) Inventors: Torbjorn Lundstedt, Uppsala (SE); Anna Skottner, Ekero (SE); Elisabeth Seifert, Uppsala (SE); Igor Starchenkov, Riga (LV); Peteris Trapencieris, Riga (LV); Valerjans Kauss, Riga (LV); Ivars Kalvins, Salaspils (LV); Arne Boman, Uppsala (SE)

Correspondence Address: STERNE, KESSLER, GOLDSTEIN & FOX PLLC, 1100 NEW YORK AVENUE, N.W., WASHINGTON, DC, 20005, US

(21) Appl. No.: 10/182,192

(22) PCT Filed: Jan. 29, 2001
(86) PCT No.: PCT/GB01/00346

(30) Foreign Application Priority Data
Jan. 28, 2000 (GB) 0001948.9
Jan. 28, 2000 (GB) 0002060.2

(51) Int. Cl. A61K 31/537; A61K 31/495; A61K 31/45; A61K 31/165; A61K 31/405; A61K 31/4035

(52) U.S. Cl. 514/237.8; 514/252.12; 514/329; 514/357; 514/419; 514/417; 514/408; 514/613; 514/625; 514/617; 544/167; 544/399; 546/233; 546/336; 548/470; 548/495; 548/567; 564/161; 564/189

(57) ABSTRACT

The present invention relates to novel aromatic amines of general formula (I) and to the use of these amines for the treatment of obesity, anorexia, inflammation, mental disorders an other diseases associated with the melanocortin receptors or related systems, e.g. the melanocyte stimulating hormones.

The present invention relates to novel aromatic amines and to the use of these amines for the treatment of obesity, anorexia, inflammation, mental disorders and other diseases associated with the melanocortin receptors or related systems, e.g. the melanocyte stimulating hormones.

A number of large linear and cyclic peptides are known in the art which show high specific binding to melanocortin (MC) receptors. The agonistic and/or antagonistic properties of these peptides are also known. See for example “Melanocortin Receptor ligands and methods of using same” by Dooley, Girten and Houghten (WO99/21571). Two patent applications (WO 99/55679 and WO 99/64002) have been published which includes small molecules showing activity on the melanocortin receptors. However the compounds in the present invention are structuarlly different from the previously published melanocortin agonists, and hence the observed effects are unexpected.

One aspect of the present invention is therefore to provide low molecular weight compounds showing activity on melanocortin receptors and which may be taken up after per oral administration and which may penetrate well through the blood brain barrier.

The present invention provides novel compounds of the general formula (I):

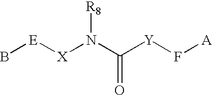

wherein E and F are independently a saturated or unsaturated, acyclic hydrocarbon group having 1, 2, 3, 4 or 5 carbon atoms.

Examples of E and F include alkyl and alkene groups, optionally substituted by one or more halogen atoms, preferably chlorine. Preferred examples of E and F include methyl, ethyl, propyl, butyl, pentyl and the corresponding alkene groups.

Wherein X and Y are independently methylene, one of X and Y are absent (i.e. it is a single bond), or X can be:

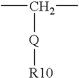

And/or Y can be:

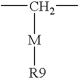

Wherein M and Q are independently a saturated or unsaturated, straight or branched chain acyclic hydrocarbon group having 1, 2, 3, 4, 5 or 6 carbon atoms; or M and/or Q are absent (i.e. M and/or Q are single bonds).

R8, R9 and R10 are independently selected from hydrogen and the following:

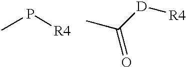

wherein P and D are independently a saturated or unsaturated, straight or branched chain acyclic hydrocarbon group having 1, 2, 3, 4, 5 or 6 carbon atoms, preferably 1, 2, 3, 4 or 5 carbon atoms; or D is absent (i.e. D is a single bond).

Examples of P and D include straight or branched chain alkyl and alkene groups, optionally substituted by one or more halogen atoms, preferably chlorine. Preferred examples of P and D include methyl, ethyl, propyl, iso-propyl, butyl, t-butyl, pentyl, t-pentyl, iso-pentyl and hexyl, and the corresponding alkene groups.

R4 is hydroxy, methyl, cyclohexyl, cyclopentyl, aminoguanidine, guanidine, carboxylic, or R4 is selected from:

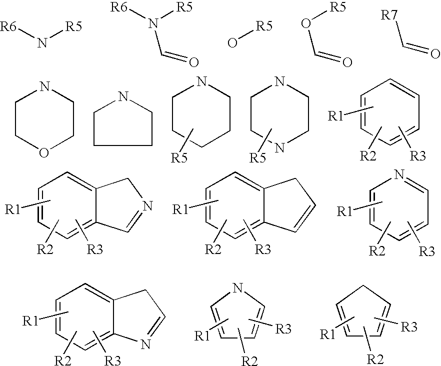

R4 in R8, R9 and R10 may be the same or different.

R5 and R6 are the same or different and are selected from hydrogen, lower alkyl such as methyl, ethyl, propyl, iso-propyl, butyl, t-butyl, pentyl, t-pentyl, iso-pentyl, cyclopropyl, cyclobutyl, cyclopentyl, cyclohexyl and hexyl.

R7 is selected from:

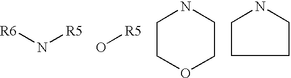

or R7 may be any one of R5 or R6.

A and B are the same or different and are selected from the following:

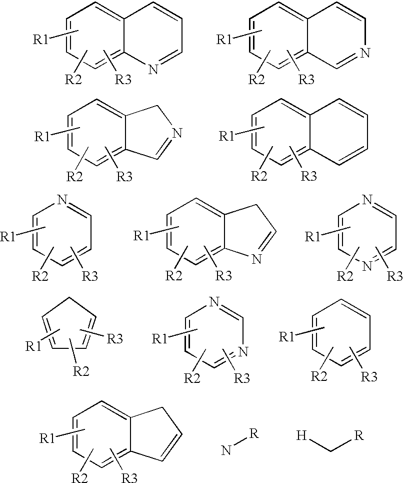

wherein R₁, R₂ and R₃ are the same or different and are selected from hydrogen, halogen, alkyl having 1 to 5 carbon atoms, electron donor groups such as alkoxy having 1-5 carbon atoms or hydroxy, electron acceptor groups selected from cyano, nitro, trifluoroalkyl or amide; and the pharmacologically active salts thereof.

Preferably, A and B are the same or different and are selected from the following:

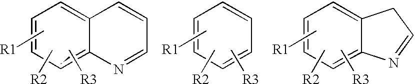

When used in the foregoing definitions, the term alkyl is meant to include straight or branched chain hydrocarbon groups; the term alkoxy is meant to include straight or branched chain alkoxy groups; and the term halogen includes fluoro, chloro or bromo.

Preferably, the “alkyl having 1 to 5 carbon atoms” is a lower alkyl such as methyl, ethyl, propyl or iso-propyl.

Preferably, the “alkoxy having 1 to 5 carbon atoms” is a lower alkoxy such as methoxy, ethoxy, propoxy or iso-propoxy.

Preferably, the halogen is fluoro or chloro.

Preferably, the trifluoroalkyl is trifluoromethyl, trifluoroethyl, trifluoropropyl or trifluoroiso-propyl.

In cases where A and/or B are bicyclic groups, it should be noted that R1, R2 and R3 represent substituents which may be present on either of the rings. Furthermore, it should be noted that A and B may be attached in the carbon backbone of the compound of general formula (I) at any suitable point within A or B, preferably at the 1, 2 or 3 position; and most preferably A and B are not attached in the carbon backbone via an N-atom in A and/or B.

The compounds of formula (I) have basic properties and, consequently, they may be converted to their therapeutically active acid addition salts by treatment with appropriate acids, e.g. inorganic acids such as hydrochloric, hydrobromic. sulphuric, nitric and phosphoric acid, or organic acids such as acetic, propanoic, glycolic, lactic, malonic, succinic, fumaric, tartaric, citric and palmoic acid.

Conversely, the salt form may be converted into the free base form by treatment with alkali.

The present invention relates novel aromatic amines. Some of the compounds of the present invention have been biologically tested in the melanocortin system and have surprisingly been shown to be capable of binding to melanocortin receptors as well as showing activity in functional assays.

Some of the compounds of the present invention are either agonists or antagonists of a specific MC-receptor or of a number of MC-receptors, e.g. MC1, MC3, MC4 or/and MC5 receptors.

The MC-receptors belong to the class of G-protein coupled receptors which are all built from a single polypeptide forming 7 transmembrane domains. Five such receptors types, termed MC1, MC2, MC3, MC4 and MC5, have been described.

The MC receptor's signaling is mainly mediated via cAMP but also other signal transduction pathways are known. They are distinctly distributed in the body.

MC-receptors are linked to a variety of physiological actions that are thought to be mediated by distinct subtypes of the MC-receptors. In many cases, however, it is not entirely clear which of the subtypes is responsible for the effect.

It has long been known that MSH-peptides may affect many different processes such as motivation, learning, memory, behaviour, inflammation, body temperature, pain perception, blood pressure, heart rate, vascular tone, brain blood flow, nerve growth, placental development, aldosterone synthesis and release, thyroxin release. spermatogenesis, ovarian weight, prolactin and FSH secretion, uterine bleeding in women, sebum and pheromone secretion, blood glucose levels, intrauterine foetal growth, as well as other events surrounding parturition (Eberle, A N: The melanotropins: Chemistry, physiology and mechanisms of action. Basel: Karger, Switzerland. 1988, ISBN 3-8055-4678-5; Gruber, and Callahan, Am. J. Physiol. 1989, 257, R681-R694; De Wildt et al., J. Cardiovascular Pharmacology. 1995. 25, 898-905), as well as inducing natriuresis (Lin et al., Hypertension. 1987, 10, 619-627).

It is also well-known that the immunomodulatory action of α-MSH includes both immuno-stimulatory and immunosuppressive effects. Several studies have shown that α-MSH antagonizes the effects of pro-inflammatory cytokines such as IL-1α, IL-1β, IL-6 and TNFα, and induces the production of the anti-inflammatory cytokine, IL-10 (for review see Catania & Lipton, 1993).

Eating behaviour is regulated by a complex network of physiological regulatory pathways that involve both the central nervous system and peripheral sites. Factors such as leptin, insulin, NPY (neuropeptide Y), orexins, CRF (Corticotropin-Releasing Factor, release hormone) and melanocortic peptides (Schwartz; Nature Medicine 1998, 4, 385-386) are known to control the amount of food intake both during short and long term, which may affect body weight, body fat mass and growth rate. Recent studies have shown a role of MC-receptors, especially the MC4 receptor, for control of food intake, and there is evidence indicating that the melanocortins and the MC4 receptor are important factors downstream of leptin. Intracerebroventricular injections of the melanocortic peptides α-MSH and ACTH(1-24) have been shown to markedly inhibit feeding (Poggioli et al., Peptides, 1986, 7, 843-848; Vergoni et al., Neuropeptides, 1986, 7. 153-158).

The MC5-receptor has recently been attributed a role in control of exocrine gland function (van der Kraan, et al., Endocrinol. 1998, 139, 2348-2355; Chen et al., Cell. 1997, 91, 789-798).

In addition, the melanocortic peptides have distinct effects on sexual functions in that they cause erection in males (Donovan, Psychol. Med. 1978, 8. 305-316), presumably mediated by a central agonistic effect of the peptide on MC-receptors. It has also been shown that a MC-receptor blocker could inhibit the erectogenic effect of melanocortic peptides (Vergoni et al., Eur. J. Pharmacol, 1998, 362; 95-101).

Some of the compounds of formula (I) and/or their pharmaceutically acceptable salts have valuable pharmacological properties, making them useful for the treatment of mental disorders such as psychoses, depression anxiety, senile dementia, Alzheimer's disease, drug abuse disorders and eating disorders such as anorexia and bulimia.

Some of the compounds of formula (I) and/or their pharmaceutically acceptable salts have valuable pharmacological properties, making them useful for the treatment of dysfunctions of the endocrine system and other hormonal systems such as excessive menstruations, endometriosis, events related to parturition, dysfunctions related to prolactin, dysfunctions related to growth hormone, dysfunctions related to testosterone, dysfunctions related to estrogen, dysfunctions related to glucocorticoids, dysfunctions related to luteinizing hormone and follicle stimulating hormone, inducing abortion, for prevention of abortion and/or for treatment of events related to parturition.

Others of the compounds of formula (I) and/or their pharmaceutically acceptable salts have valuable pharmacological properties making them useful for the treatment of sexual functions/dysfunctions such as inducing erection in man, to induce erection in animal breeding, to stimulate intercourse in animals which are difficult to mate, in particular rare species or valuable strains, pets, cats, dogs, horses or to reduce sexual behaviour in animals, e.g. for pets, cats etc., to treat impotence and disorders related to sexual drive, including lack of sexual drive or abnormal sexual drive in both men and women.

Some of the compounds of formula (I) and/or their pharmaceutically acceptable salts have valuable pharmacological properties, making them useful for the treatment of inflammation such as inflammations related to the production of nitric oxide, inflammation related to increased amounts (upregulated amounts) of inducible nitric oxide synthase, inflammation related to activation of transcriptional activators, inflammation related to nuclear factor kappa beta, inflammation related to macrophages, neutrophils, monocytes, keratinocytes, fibroblasts, melanocytes, pigment cells and endothelial cells, inflammation related to increased production and/or release of inflammatory cytokines, such as e.g. interleukins, in particular interleukin 1 (IL-1), interleukin 6 (IL-6) and tumor necrosis factor α (TNF-α).

In the present specification, “increased production” refers to increased formation, increased release, or increased amount of an endogenous compound locally, regionally or systemically in a patient compared to the amount of said endogenous compound in a healthy individual. In the present specification, “upregulated” refers to an increased activity or amount of the compound compared with that in a healthy individual.

In the present specification, “decreased production” refers to decreased formation, decreased release, or decreased amount of an endogenous compound in a patient compared to the amount of said endogenous compound in a healthy individual. In the present specification, “downregulated” refers to a decreased activity, or amount of the compound compared with that in a healthy individual.

In particular, positive treatment effects or preventive effects may be seen in conditions where inflammation or an inflammatory-like condition is caused by or being associated with one or more of the following: allergy, hypersensitivity, bacterial infection, viral infection, inflammation caused by toxic agent, fever, autoimmune disease, radiation damage by any source including UV-radiation, X-ray radiation, γ-radiation, α- or β-particles, sun bums, elevated temperature or mechanical injury. Moreover, inflammation due to hypoxia, which is optionally followed by reoxygenation of the hypoxic area, is typically followed by severe inflammation, which condition may be positively affected by treatment with a compound of the invention.

In very specific embodiments of the invention, a compound of the invention may be administered for the prevention or therapeutic treatment of inflammatory diseases of the skin (including the dermis and epidermis) of any origin, including skin diseases having an inflammatory component. Specific examples of this embodiment of the invention include treatment of contact dermatitis of the skin, sunburns of the skin, burns of any cause, and inflammation of the skin caused by chemical agents, psoriasis, vasculitis, pyoderma gangrenosum, discoid lupus erythematosus, eczema. pustulosis palmo-plantaris, and phemphigus vulgaris.

Also comprised by the invention is the administration of a compound of formula (I) or a pharmacologically acceptable salt thereof for the treatment of an inflammatory disease in the abdomen, including an abdominal disease having an inflammatory component. Specific examples of the treatment of such a disease with a compound of the invention are gastritis, including one of unknown origin, gastritis perniciosa (atrophic gastritis), ulcerous colitis (colitis ulcerosa), morbus Crohn, systemic sclerosis, ulcus duodeni, coeliac disease, oesophagitis and ulcus ventriculi.

Comprised by the invention is also the administration of a compound of formula (I) or a pharmacologically acceptable salt thereof for the treatment of systemic or general and/or local immunological diseases, including those of an autoimmune nature, and other inflammatory diseases of a general nature. Specific examples include treatment of rheumatoid arthritis, psoriatic arthritis, systemic sclerosis, polymyalgia rheumatica, Wegener's granulomatosis, sarcoidosis, eosinophilic fasceitis, reactive arthritis, Bechterew's disease, systemic lupus erythematosus, arteritis temporalis. Behcet's disease, morbus Burger, Good Pastures' syndrome, eosinophilic granuloma, fibromyalgia, myositis and mixed connective tissue disease. Included therein is also arthritis, including arthritis of unknown origin.

Further included in the invention is administration of a compound of formula (I) or a pharmacologically acceptable salt thereof for the treatment of a disease of the peripheral and/or central nervous system related to inflammation. Included in this aspect of the invention is the treatment of cerebral vasculitis, multiple sclerosis, autoimmune ophthalmitis and polyneuropathia. Comprised by the invention is also the administration of a compound of the invention for the treatment of an inflammation of the central nervous system to prevent apoptotic cell death. Moreover, as some of the compounds of the invention show a distinct ability to induce nerve regeneration, positive treatment effects are often seen in central nervous system diseases involving damage of cells in this region. This aspect of the invention also includes treatment of traumatic injuries to the central nervous system, brain edema multiple sclerosis, Alzheimer's disease, bacterial and viral infections in the central nervous system, stroke, and haemorrhagia in the central nervous system.

Comprised by the invention is also the administration of a compound of formula (I) or a pharmacologically acceptable salt thereof for the treatment of diseases of the eye and tear glands related to inflammation. Specific examples of such diseases comprise anterior and posterior uveitis, retinal vasculitis, optic neuritis, optic neuromyelitis, Wegener's granulomatosis, Sjogren's syndrome, episcleritis, scieritis, sarcoidosis affecting the eye and polychondritis affecting the eye.

Comprised by the invention is also the administration of a compound of formula (I) or a pharmacologically acceptable salt thereof for the treatment of diseases of the ear related to inflammation, specific examples of which include polychondritis affecting the ear and external otitis.

Comprised by the invention is also the administration of a compound of formula (I) or a pharmacologically acceptable salt thereof for the treatment of diseases of the nose related to inflammation, specific examples of which are sarcoidosis, polychondritis and mid-line granuloma of the nose.

Comprised by the invention is also the administration of a compound of formula (I) or a pharmacologically acceptable salt thereof for the treatment of diseases related to inflammation of the mouth, pharynx and salivary glands. Specific examples include Wegener's granulomatosis, mid-line granuloma, Sjogren's syndrome and polychondritis in these areas.

Included in the invention is also the administration of a compound of formula (I) or a pharmacologically acceptable salt thereof for the treatment of diseases related to inflammation in the lung. Specific examples include treatment of idiopathic alveolitis, primary pulmonary hypertension, bronchitis, chronic bronchitis, sarcoidosis, alveolitis in inflammatory systemic disease, pulmonary hypertension in inflammatory systemic disease, Wegener's granulomatosis and Good Pastures' syndrome.

Comprised by the invention is also the administration of a compound of formula (I) or a pharmacologically acceptable salt thereof for the treatment of diseases related to the inflammation of the heart. Specific examples include treatment of pericarditis, idiopathic pericarditis, myocarditis, Takayasus' arteritis, Kawasaki's disease, coronary artery vasculitis, pericarditis in inflammatory systemic disease, myocarditis in inflammatory systemic disease, endocarditis and endocarditis in inflammatory systemic disease.

Comprised by the invention is also the administration of a compound of formula (I) or a pharmacologically acceptable salt thereof for the treatment of diseases related to inflammation of the liver. Specific examples include treatment of hepatitis, chronic active hepatitis, biliary cirrhosis, hepatic damage by toxic agents, interferon induced hepatitis, hepatitis induced by viral infection, liver damage induced by anoxia and liver damage caused by mechanical trauma.

Comprised by the invention is also the administration of a compound of formula (I) or a pharmacologically acceptable salt thereof for the treatment of diseases related to inflammation of the pancreas. Specific examples include treatment (and prevention) of diabetes mellitus, acute pancreatitis and chronic pancreatitis.

Comprised by the invention is also the administration of a compound of formula (I) or a pharmacologically acceptable salt thereof for the treatment of diseases related to the inflammation of the thyroidea. Specific examples of these embodiments of the invention include treatment of thyreoiditis, autoimmune thyreoiditis and Hashimoto's thyreoiditis.

Comprised by the invention is also the administration of a compound of formula (I) or a pharmacologically acceptable salt thereof for the treatment of diseases related to inflammation of the kidney. Specific examples include treatment of glomerulonephritis, glomerulonephritis in systemic lupus erythematosus, periarteritis nodosa, Wegener's granulomatosis, Good-Pastures'syndrome, HLAb27 associated diseases, IgA nephritis (IgA=Immunoglobulin A), pyelonephritis. chronic pyelonephritis and interstitial nephritis.

Comprised by the invention is also the administration of a compound of formula (I) or a pharmacologically acceptable salt thereof for the treatment of diseases related to the inflammation of the joints. Specific examples include treatment of Bechterew's disease, psoriatic arthritis, rheumatoid arthritis, arthritis in colitis ulcerosa, arthritis in morbus Crohn, affection of joints in systemic lupus erythematosus, systemic sclerosis, mixed connective tissue disease, reactive arthritis, Reiter's syndrome. Moreover, included in this embodiment of the invention is treatment of arthrosis of any joint, in particular arthrosis of finger joints, the knee and the hip.

Comprised by the invention is also the administration of a compound of formula (I) or a pharmacologically acceptable salt thereof for the treatment of diseases related to the inflammation of blood vessels. Specific examples include treatment of arteritis temporalis, periarteritis nodosa, arteriosclerosis, Takayasus' arteritis and Kawasaki's disease. Particularly advantageous is the capacity of some compounds of the invention to afford protection against and prevention of arteriosclerosis. This is in part due to the capacity of some compounds of formula (I) or the pharmacologically acceptable salts thereof to prevent the induction of inducible nitric oxide synthesis (iNOS) caused by the action of oxidized Low Density Lipoprotein on endothelial cells and blood vessel walls.

Comprised by the invention is also the administration of a compound of the invention for the treatment of drug-induced disorders of the blood and lymphoid system, including the treatment of drug-induced hypersensitivity (including drug hypersensitivity) affecting blood cells and blood cell forming organs (e.g. bone marrow and lymphoid tissue). Specific embodiments of this aspect of the invention include the treatment of anemia, granulocytopenia, thrombocytopenia, leukopenia, aplastic anemia, autoimmune hemolytic anemia, autoimmune thrombocytopenia and autoimmune granulocytopenia.

The compounds of the invention may also be administered for the treatment of fast allergic disorders (Type I allergy). Included in this embodiment of the invention is the treatment of anaphylactic reactions, anaphylactoid reactions, asthma, asthma of allergic type, asthma of unknown origin, rhinitis, hay fever and pollen allergy.

Comprised by the invention is also the administration of a compound of formula (I) or a pharmacologically acceptable salt thereof for the treatment of inflammation related to infections of any origin. Specific examples include treatment of inflammation secondary to infection caused by virus, bacteria, helminths and protozoae.

Comprised by the invention is also the administration of a compound of formula (I) or a pharmacologically acceptable salt thereof for the treatment of inflammations related to trauma and/or tissue injury of any origin.

Some of the compounds of formula (I) or pharmaceutically acceptable salts thereof have valuable pharmacological properties, making them useful for the treatment of disorders of the cardiovascular system such as disorders related to blood pressure, heart rate, vascular tone, natriuresis, bleeding, shock, disorders related to ischemia, infarction, repercussion injuries, arrhythmias of the heart, in particular during ischemia, or for the treatment of arrhythmias associated with reoxygenation of a previously ischemic period of the heart.

Some of the compounds of formula (I) or the pharmaceutically acceptable salts thereof have valuable pharmacological properties, making them useful for the treatment of pain such as pain of central origin, pain seen after damage to the CNS, stroke, infarction, pain of peripheral origin, chronic pain, neuropathies and disorders where a treatment effect is achieved by stimulation of receptors in the periaqueductal grey area.

Because of the capacity of some of the compounds of the invention to stimulate pigment formation in epidermal cells, some of the compounds of the invention may be also useful for inducing skin tanning for cosmetic reasons, for treatment of vitiligo, or any other condition where darkening of skin color is desired. Moreover, because of the ability of some of the compounds of the invention to inhibit pigment formation in cells of the skin, they may also be useful for inducing lighter skin color for cosmetic reasons, or during any condition where a lighter color of skin is desired.

Some of the compounds of formula (I) or the pharmaceutically acceptable salts thereof have valuable pharmacological properties, making them useful to cause skin tanning, darkening the colour of the skin, to induce melanin synthesis in the skin, to reduce skin tanning, lightening the colour of the skin, to reduce or block melanin synthesis in the skin, to cause anti-inflammatory actions in the skin, to modulate epidermal growth, to improve wound healing, to treat acne, seborrhoea, acne roseacea conditions related to malfunctions of the glands of the skin, e.g. sebacous glands and over or underproduction of sebum.

Some of the compounds of the invention are useful for inhibiting or stimulating the in vivo formation of second messenger elements such as cAMP. Such inhibition/stimulation may be used in cells or crushed cell systems in vitro. e.g. for analytical or diagnostic purposes.

For analytical and diagnostic purposes the compounds of the invention may be used in radioactive form where they comprise one or more radioactive labels or gamma or positron emitting isotopes, to be used in radioligand binding for the quantification as well as tissue localisation of MC-receptors, for analysis of dissociation/association constants, and for imaging of in vivo binding by the use of scintigraphy, positron emission tomography (PET) or single photon emission computed tomography (SPECT), or for the diagnosis of disease and treatment of any malignancy where the malignant cells contain MC receptors.

Alternatively the compounds of the invention can be labelled with any other type of label that allows detection of the respective compound, e.g. fluorescence, biotin, or labels activated by gamma-irradiation, light photons or biochemical processes. or by light or UV-light (the latter in order to obtain a compound useful for covalent labelling of MC receptors by a photoaffinity technique).

Some of the compounds of formula (I) or the pharmacologically acceptable salts thereof may also be tagged with a toxic agent (i.e. doxorubicin, ricin, diphtheria toxin or other) and used for targeted delivery to malignant cells bearing MC receptors, or tagged with a compound capable of activating the endogenous immune system for triggering the immune system (for example a compound. monoclonal antibody or other, capable of binding to a T-cell antigen, e.g. CD3 or other) for treatment of malignancies and other MC receptor expressing diseases. The thus formed hybrid compound will direct cytotoxic cells to the malignant melanoma cells or the MC1-receptor bearing malignant cells and inhibit the tumor growth.

Some of the compounds of formula (I) or a pharmacologically acceptable salt thereof may be attached to the antibody chemically by covalent or non-covalent bond(s).

Some of the compounds of the invention may be used for the treatment and diagnosis of diseases, disorders and/or pathological conditions in an animal, in particular in man.

The present invention also relates to a pro-drug which, upon administration to an animal or a human, is converted to a compound of the invention. Pro-drugs of the compounds of formula (I) and their pharmacologically acceptable salts may be used for the same purposes as described in this specification for the compounds of the invention, as well as is disclosed in the Examples given below.

The compounds of the present invention may be bound covalently or non-covalently to one or several of other molecule(s) of any desired structure(s); the thus formed modified compound or complex may be used for the same purposes as described in this specification for the compounds of the invention, as well as is disclosed in the Examples given below. In a particularly important embodiment of the invention, a radioactively-labeled molecule is covalently bound to a compound of formula (I) or a pharmacologically acceptable salt thereof so as to make a compound of formula (I) or a pharmacologically acceptable salt thereof radioactively labeled.

The invention also relates to methods for the manufacture and pharmaceutical preparations comprising one or more of the compounds of the invention, as well as to their uses for various medical and veterinary practices related to melanocyte stimulating hormone receptors.

Some of the compounds of the invention bind to one or more MC-receptors. By the term “bind to one or more MC-receptors” is in this context intended a capacity of the compound of the invention to compete for the binding of [¹²⁵I]-NDP-MSH at an MC-receptor the MC-receptor preferably being one selected from the MC1, MC3, MC4 and/or MC5-receptors, using a binding assay such as that described in Example 3. In a further meaning, the term “bind to one or more MC-receptors” is in this context intended that the Ki-value of the compound of the invention, determined using a method such as that described in Example 3, is less than 1,000,000 nM, preferably less than 100,000 nM, more preferably less than 10,000 nM, somewhat more preferably less than 1,000 nM, even somewhat preferably less than 100 nM, and most preferably less than 50 nM. Most preferably, the compound of the invention has a Ki of less than 1,000 nM or less than 50 nM for a melanocortin receptor.

The compounds having the general formula (I) may be prepared by using standard procedures. Reference may also be made in this regard to the following Examples.

### LEGENDS TO THE FIGURES

FIGS. 1-4 In vivo effects on food intake and body weight gain.

FIGS. 5-6 In vivo effects of Compound 2:7 on paw oedema and total number of white blood cells.

FIGS. 7-8 In vivo effects of Compound 1:15 on paw oedema and total number of white blood cells.

FIGS. 9-10 In vivo effects of Compound 1:17 on paw oedema and total number of white blood cells.

### EXAMPLES

The following examples are intended to illustrate but not to limit the scope of the invention, although the compounds named are of particular interest for the intended purposes. These compounds have been designated by a number code, a:b, where a means the number of the example where the preparation of the compound is described, and b refers to the order of the compound prepared according to that example. Thus example 1:2 means the second compound prepared according to example 1.

The structures of the compounds were confirmed by IR, NMR, MS and elementary analysis. When melting points are given, these are uncorrected.

### Example 1:1

N-[1-Benzyl-2-(4-carbamimidoyl-piperazin-1-yl)-2-oxo-ethyl]-3-(1H-indol-3-yl)-propionamide

(Benzyloxycarbonylimino-piperazin-1-yl-methyl)-carbamic acid benzyl ester

To a suspension of piperazine hexahydrate (2.33 g, 12 mmol) in acetonitrile (30 ml) 1,3-bis-benzyloxycarbonyl-2-methylthiopseudourea (3.58 g, 10 mmol) was added at room temperature and stirred for 8 h, the precipitate was filtered off, washed with acetonitrile and water, dried (P₂O₅ and NaOH) to give the product (3.37 g, 85%) as colourless foam. ¹H NMR (CDCl₃, TMS), δ2.89 (4H, t, J=5.1 Hz); 3.57 (4H, m); 5.13 (4H, s); 7.24-7.42 ppm (10H, m).

[1-Benzyl-2-(4-carbamimidoyl-piperazin-1-yl)-2-oxo-ethyl]-carbamic acid tert-butyl ester

To a cooled suspension of (benzyloxycarbonylimino-piperazin-1-yl-methyl)-carbamic acid benzyl ester (0.79 g, 2 mmol), N-hydroxysuccinimide (0.23 g, 2 mmol), N-butyloxycarbonyl-phenylalanine (0.53 g, 2 mmol) in CH₂Cl₂ (20 ml) at 0° C. was added dicyclohexyl-carbodiimide (0.43 g, 2.1 mmol). The reaction mixture was stirred at 0° C. for 4 h, filtered at the same temperature, washed with NaHCO₃ solution, water and brine, dried (MgSO₄), evaporated in vacuo and purified by chromatography (silica gel; petroleum ether-ethylacetate, 2:1) to give the product (0.27 g, 42%) as a colourless foam. ¹H NMR (CDCl₃, TMS), δ1.41 (9H, s); 2.47-3.87 (10H, m); 5.11 (4H, s); 5.23-5.48 (1H, m); 7.07-7.61 (16H, m); 10.49 ppm (1H, br s).

N-[1-Benzyl-2-(4-carbamimidoyl-piperazin-1-yl)-2-oxo-ethyl]-3-(1H-indol-3-yl)-propionamide

A solution of [1-Benzyl-2-(4-carbamimidoyl-piperazin-1-yl)-2-oxo-ethyl]-carbamic acid tert-butyl ester (0.21 g, 0.32 mmol) in 98% formic acid (4ml) was kept for 12 h at room temperature, acid evaporated in vacuo at 30° C., dissolved in acetonitrile (10 ml). Activated ester of indolylpropionic acid (0.09 g, 0.33 mmol) and NaHCO₃ solution until pH 8-10 were added consequently, stirred for 8 h at room temperature, evaporated in vacuo, dissolved in ethylacetate (12 ml). The organic layer was washed with water, brine, dried (MgSO₄), and then evaporated in vacuo. The crude residue was dissolved in ethanol (5 ml) and hydrogenated by the method of example 2 to give after chromatography (silica gel; chloroform-methanol-water, 100:20:1) the product (0.063 g, 40%) as a foam. ¹H NMR (D₂O, TSS), δ2.49-3.71 (15H, m); 6.96-7.69 ppm (10H, m). Anal. Calcd for C₂₅H₃₀N₆O₂*HCl*0.5H₂O: C 61.03; H 6.56;N 17.08. Found: C 61.35; H 6.61; N 16.83.

The following compounds may be made in a similar manner.

| | | | Melt. |
|---|---|---|---|
| No. | Compound name | Salt | Point |
| 1:2 | N-Cyclohexyl-2-[[2-(1H-indol-3-yl)-ethyl]-(2-naphthalen- | | 212-213 |
| | 1-yl-acetyl)-amino]-2-naphthalen-1-yl-acetamide | | |
| 1:3 | 1H-Indole-2-carboxylic acid [(4-cyano-phenyl)- | | 130-135 |
| | cyclohexylcarbamoyl-methyl]-[2-(1H-indol-3-yl)-ethyl]- | | dec. |
| | amide | | |
| 1:4 | N-Cyclohexyl-2-{(2-1H-indol-3-yl-acetyl)-[2-(1H-indol-3- | | 125-130 |
| | yl)-ethyl]-amino}-2-pyridin-3-yl-acetamide | | |
| 1:5 | 2-(4-Chloro-phenyl)-N-cyclohexyl-2-[[2-(1H-indol-3-yl)- | | 88-90 |
| | ethyl]-(2-naphthalen-2-yl-acetyl)-amino]-acetamide | | |
| 1:6 | N-(3-Amino-propyl)-3-(1H-indol-3-yl)-2-(2-naphthalen-1- | 1.2 | 130-135 |
| | yl-acetylamino)-propionamide | HCl,H2O | (fish) |
| 1:7 | N-(3-Amino-propyl)-3-(1H-indol-3-yl)-2-(2-naphthalen-2- | 1.2 | 137-140 |
| | yl-acetylamino)-propionamide | HCl, H2O | (fish) |
| 1:8 | N-(3-Amino-propyl)-3-(1H-indol-3-yl)-2-(2-1H-indo]-3- | 2.2HCl, H | 143-148 |
| | yl-acetylamino)-propionamide | 2O, 0.5CH | (fish) |
| | | 3CN | |
| 1:9 | N-(3-Guanidino-propyl)-3-(1H-indol-3-yl)-2-(2- | 1.25 HCl, | 134-136 |
| | naphthalen-2-yl-acetylamino)-propionamide | H2O | (fish) |
| 1:10 | N-(3-Amino-propyl)-3-(1H-indol-3-yl)-2-(3-1H-indol-3- | 1.5 HCl, | 140-145 |
| | yl-propionylamino)-propionamide | H2O | (fish) |
| 1:11 | N-[1-(3-Amino-propylcarbamoyl)-2-(1H-indol-3-yI)- | 2 HCl, | 135-140 |
| | ethyl]-4-(1H-indol-3-yl)-butyramide | H2O | (fish) |
| 1:12 | N-(3-Guanidino-propyl)-3-(1H-indol-3-yl)-2-(2-1H-indol- | HCl, H2O | 132-137 |
| | 3-yl-acetylamino)-propionamide | | (fish) |
| 1:13 | N-Cyclohexyl-2-[[2-(1H-indol-3-yl)-ethyl]-(3-phenyl- | 0.5H2O | |
| | propionyl)-amino]-4-phenyl-butyramide | | |
| 1:14 | N-Benzy1-N-4-guanidino-butyl)-3-(1H-indol-3-yl)-2-(2- | Flav | 142-150 |
| | 1H-indol-3-yl-acetylamino)-propionamide | | (fish) |
| 1:15 | N-[1-[Benzyl-(4-guanidino-butyl)-carbamoyl]-2-(1H-indol- | HCl, H2O | 120-125 |
| | 3-yl)-ethyl]-4-phenyl-butyramide | | (fish) |
| 1:16 | 3-Benzo[1,3]dioxol-5-yl-N-[1-[benzyl-(4-guanidino- | HCl, H2O | 127-131 |
| | butyl)-carbamoyl]-2-(1H-indol-3-yl)-ethyl]-propionamide | | (fish) |
| 1:17 | N-Benzyl-N-(4-guanidino-butyl)-3-(1H-indol-3-yl)-2-(2- | HCl, H2O | 129-133 |
| | naphthalen-2-yl-acetylamino)-propionamide | | (fish) |
| 1:18 | N-[(4-Cyano-phenyl)-cyclohexylcarbamoyl-methyl]-N-(2- | HCl | |
| | dimethylamino-ethyl)-3-(1H-indol-3-yI)-propionamide | | |
| 1:19 | N-Benzhydryl-2-(2-methyl-1H-indol-3-yl)-acetamide | | 135-137 |
| 1:20 | N-(1,2-Diphenyl-ethyl)-4-(1H-indol-3-yL)-butyramide | HCl? | 134 |
| 1:21 | N-(1,2-Diphenyl-ethyl)-2,6-dimethoxy-nicotinamide | | 116-118 |
| 1:22 | N-Benzhydryl-2,6-dimethoxy-nicotinamide | | 123-126 |
| 1:23 | 2-(2-Bromo-phenyl)-N-cyclohexyl-2-[(2-dimethylamimo- | | — |
| | ethyl)-(2-1H-indol-3-yl-acetyl)-aminol-acetamide | | |
| 1:24 | 2-[[2-(5-Bromo-1H-indol-3-yl)-acetyl]-(2-dimethylamino- | HCl | — |
| | ethyl)-amino]-2-(2-bromo-phenyl)-N-cyclohexyl- | | |
| | acetamide | | |
| 1:25 | N-Benzhydryl-2-chloro-6-methyl-nicotinamide | | 189 |

### Example 2:1

4-Amino-N-[2-(1H-indol-3-yl)-1-(3-phenyl-propylcarbamoyl)-ethyl]-butyramide hydrochloride

4-Benzyloxvcarbonylamino-butyric acid 2,5-dioxo-pyrrolidin-1-yl ester

A solution of 4-benzyloxycarbonylamino-butyric acid (14.22 g, 60 mmol) and N-hydroxysuccinimide (6.90 g, 60 mmol) in acetonitrile (80 ml) at 0° C. was treated with dicyclohexylcarbodiimide (13.60 g, 66 mmol). The resulting suspension was kept for 2 days at 0° C., precipitate was filtered off and washed with ethylacetate (3×30 ml). Solvents were removed in vacuo and the residue crystallised from isopropanol (40 ml) to give the activated ester (18.9 g, 91%) as a colourless crystals. ¹H NMR (CDCl₃, TMS), δ1.74-2.12 (2H, m); 2.64 (2H, t, J=7.3 Hz): 2.77 (4H, s); 3.28 (2H, q, J=7.2 Hz); 5.09 (3H, s); 7.23-7.48 ppm (5H, m).

(4-Benzyloxycarbonylamino-butyrylamino)-(1H-indol-3-yl)-acetic acid

A suspension of L-tryptophan (0.61 g, 3 mmol) and NaHCO₃ (0.50 g, 6 mmol) in water (10 ml) was treated with 4-benzyloxycarbonylamino-butyric acid 2,5-dioxo-pyrrolidin-1-yl ester (1.04 g, 3 mmol). Acetonitrile (˜5 ml) was added to get a clear solution. After stirring for 12 h, the reaction mixture was concentrated in vacuo to give an oil, acidified with citric acid to pH2, washed twice with water, dissolved in ethylacetate, washed with water (2×20 ml), brine (2×20 ml), and dried (MgSO₄). After concentration in vacuo the crude amide (1.25 g, 95%) was isolated. ¹H NMR (CDCl₃, TMS), δ1.37-2.14 (4H, m); 2.65-3.44 (4H, m); 4.69-5.29 (3H, m); 6.40-7.64 (12H, m); 8.05 (1H, br s); 10.18 ppm (1H, br s).

4-Amino-N-[2-(1H-indol-3-yl)-1-(3-phenyl-propylcarbamoyl)-ethyl]-butyramide

A suspension of crude (4-Benzyloxycarbonylamino-butyrylamino)-(1H-indol-3-yl)-acetic acid (0.42 g, 1 mmol), N-hydroxysuccinimide (0.12 g, 1 mmol) and phenylpropylamine in CH₂Cl₂ (7 ml) at 0° C. was treated with dicyclohexylcarbodiimide (0.23 g, 1.1 mmol). After stirring for 12 h, the precipitate was filtered off and washed with CH₂Cl₂ (5 ml). The organic layer was washed with saturated NaHCO₃ solution, water and brine, dried (MgSO₄), concentrated in vacuo, and the residue purified by column chromatography (silica gel; under a concentration gradient acetonitrile—acetonitrile-water, 21:1). The thus obtained intermediate product was dissolved in warm ethanol (40 ml), 5% Pd/C (70 mg) and 5 drops of conc HCl were added and the reaction mixture was hydrogenated for 1 h at an ambient pressure. The Pd catalyst was filtered off, the solution was concentrated in vacuo, the residue purified by column chromatography (silica gel; chloroform-methanol-water, 100:20:1) and dried (P₂O₅, then NaOH) to give the title product (0.28 g; 61%) as a foam. ¹H NMR spectrum (DMSO-D6, TMS), δ1.45-1.82 (m, 4H); 2.10-2.35 (m, 2H); 2.60-2.80 (m, 2H); 2.85-3.20 (m, 4H); 3.904.25 (m, 2H); 4.54 (q, 1H, J=5.4Hz); 6.98-7.40 (m, 9H); 7.66 (d, 1H, J=7.6 Hz); 8.05-8.32 (m,4H); 8.27 (d,1H, J=8.2 Hz); 10.96 ppm (s, 1H). Anal. Calcd for C₂₄H₃₀N₄O₂*HCl*3%NaCl: C 62.6; H 6.8; N 12.2. Found: C 62.0; H 6.9; N 12.0.

The following compounds were prepared in a similar manner:

| Compound | Compound | | Melting |
|---|---|---|---|
| Number | Name | Salt | Point |
| 2:2 | 4-Guanidino-N-[2-(1H-indol-3-yl)-1- | HCl, H2O | 110-112 |
| | nethylcarbamoyl-ethyl]-butyramide | | |
| 2:3 | 4-Guanidino-N-[2-(1H-indol-3-yl)-1-(3-phenyl- | HCl, H2O | 110-112 |
| | propylcarbamoyl)-ethyl]-butyramide | | |
| 2:4 | 4-Guanidino-N-{2-(1H-indol-3-yl)-1-[2-(1H-indol-3- | HCl | 144-157 |
| | yl)-ethylcarbamoyl]-ethyl}-butyramide | | (Fish) |
| 2:5 | 4-Guanidino-N-{2-(1H-indol-3-yl)-1-{2-(1H-indol-3- | 0.8*Flav | 190-203 |
| | yl)-ethylcarbamoyl]-ethyl}-butyramide | +H2O | (fish) |
| 2:6 | N-[1-(9-Ethyl-9H-carbazol-3-ylcarbamoyl)-2-(1H- | HCl, | 163-166 |
| | indol-3-yl)-ethyl]-4-guanidino-butyramide | 2H2O | (fish) |
| 2:7 | 4-Amino-N-[1-(9-ethyl-9H-carbazol-3- | HCl. H2O | 182-187 |
| | ylcarbamoyl)-2-(1H-indol-3-yI)-ethyl]-butyramide | | (fish) |
| 2:8 | 4-Amino-N-[2-(1H-indol-3-yl)-1-(naphthalen-2- | HCl. H2O | 192-198 |
| | ylcarbamoyl)-ethyl]-butyramide | | (fish) |
| 2:9 | N-[2-(1H-Indol-3-yl)-1-(naphthalen-2-ylcarbamoyl)- | | 110-112 |
| | ethyl]-4-(3-methyl-thioureido)-butyramide | | (fish) |
| 2:10 | 2-(3-Guanidino-propionylamino)-3-(1H-indol-3-yl)- | 1.3HCl, | 130-135 |
| | N-[2-(1H-indol-3-yl)-ethyl]-propionamide | H2O | |
| 2:11 | 4-Amino-N-[1-(9-ethyl-9H-carbazol-3- | HCl. H2O | |
| | ylcarbamoyl)-2-(5-hydroxy-1H-indol-3-yl)-ethyl]- | | |
| | butyramide | | |
| 2:12 | 4-Amino-N-[2-(1H-indol-3-yl)-1-(4-phenyl- | HCl. H2O | 85-88 |
| | butylcarbamoyl)-ethyl]-buryramide | | |
| 2:13 | 4-Amino-N-[2-(5-hydroxy-1H-indol-3-yl)-1-(4- | HCl. H2O | |
| | phenyl-butylcarbamoyl)-ethyl]-butyramide | | |
| 2:14 | 2-(3-Amino-propionylamino)-N-(9-ethyl-9H- | HCl. H2O | 170-173 |
| | carbazol-3-yl)-3-(1H-indol-3-yl)-propionamide | | |
| 2:15 | 4-Amino-N-[2-(5-hydroxy-1H-indol-3-yl)-1- | HCl. H2O | |
| | phenethylcarbamoyl-ethyl]-butyramide | | |
| 2:16 | 4-Amino-N-[1-[2-(3,4-dimethoxy-phenyl)- | HCl. H2O | |
| | ethylcarbamoyl]-2-(5-hydroxy-1H-indol-3-yl)- | | |
| | ethyl]-butyramide | | |
| 2:17 | 4-Amino-N-[2-(5-methyl-1H-indol-3-yl)-1- | HCl, H2O | |
| | phenethylcarbamoyl-ethyl]-butyramide | | |
| 2:18 | 2-(3-Amino-propionylamino)-3-(1H-indol-3-yl)-N- | HCl, H2O | |
| | phenethyl-propionamide | | |
| 2:19 | 2-(3-Amino-propionylamino)-3-(1H-indol-3-yl)-N- | HCl. H2O | |
| | [2-(1H-indol-3-yl)-ethyl]-propionamide | | |
| 2:20 | 4-Amino-N-[1-benzylcarbamoyl-2-(1H-indol-3-yl)- | HCl. H2O | |
| | ethyl]-butyramide | | |
| 2:21 | Piperidine-4-carboxylic_acid_[1-(9-ethyl-9H- | 2 HCl | |
| | carbazol-3-ylcarbamoyl)-2-(1H-indol-3-yl)-ethyl]- | | |
| | amide | | |
| 2:22 | Piperidine-4-carboxylic_acid_{2-(1H-indol-3-yl)-1- | HCl. H2O | |
| | [2-(1H-indol-3-yl)-ethylcarbamoyl]-ethyl}-amide | | |
| 2:23 | 2-(3-Amino-propionylamino)-N-[2-(3,4-dimethoxy- | HCl. H2O | 151-153 |
| | phenyl)-ethyl]-3-(1H-indol-3-yl)-propionamide | | |
| 2:24 | Piperidine-4-carboxylic_acid_[2-(1H-indol-3-yl)-1- | HCl, H2O | |
| | phenethylcarbamoyl-ethyl]-amide | | |
| 2:25 | 2-(3-Amino-propionylamino)-3-(1H-indol-3-yl)-N- | HCl. H2O | |
| | (3-phenyl-propyl)-propionamide | | |
| 2:26 | 4-Amino-N-[1-[(benzo[1,3]dioxol-5-ylmethyl)- | HCl, H2O | 136-138 |
| | carbamoyl]-2-(1H-indol-3-yl)-ethyl]-butyramide | | |
| 2:27 | 2-(3-Ainino-propionylamino)-3-(1H-indol-3-yl)-N- | HCl. H2O | |
| | (4-phenyl-butyl)-propionamide | | |
| 2:28 | 4-Amino-N- [2-(1H-indol-3-yl)-1-(quinolin-4- | 2HCl, | |
| | ylcarbamoyl)-ethyl]-butyramide | H2O | |
| 2:29 | 4-Amino-N-[2-(1H-indol-3-yl)-1-(pyridin-2- | 2HCl ,H2O | 172-175 |
| | ylcarbamoyl)-ethyll-butyramide | | |
| 2:30 | 4-Amino-N[1-(indan-2-ylcarbamoyl)-2-(1H-indol- | HCl, H2O | |
| | 3-yl)-ethyl]-butyramide | | |
| 2:31 | 4-Amino-N-[2-(1H-indol-3-yl)-1-(3,4,5-trimethoxy- | HCl, H2O | |
| | benzylcarbamoyl)-ethyl]-butyramide | | |
| 2:32 | 4-Amino-N-{2-(1H-indol-3-yl)-1-[(naphthalen-2- | HCl, H2O | |
| | ylmethyl)-carbamoyl]-ethyl}-butyramide | | |
| 2:33 | 4-Amino-N-[1-(1,2-diphenyl-ethylcarbamoyl)-2- | HCl, H2O | |
| | (1H-indol-3-yl)-ethyl]-butyramide | | |
| 2:34 | 4-Amino-N-[2-(1H-indol-3-yl)-1-(pyridin-3- | 2HCl ,H2O | |
| | ylcarbamoyl)-ethyl]-butyramide | | |
| 2:35 | 4-Amino-N-[2-(1H-indol-3-yl)-1-(quinolin-6- | 2HCl ,H2O | |
| | ylcarbamoyl)-ethyl]-butyramide | | |
| 2:36 | 4-Amino-N-[2-(1H-indol-2-yl)-1-(4-trifluoromethyl- | | — |
| | phenylcarbamoyl)-ethyl]-butyramide | | |
| 2:37 | 4-Amino-N-[1-(9-ethyl-9H-carbazol-3- | HCl | |
| | ylcarbamoyl)-2-phenyl-ethyl]-butyramide | | |
| 2:38 | 4-Amino-N-{2-(1H-indol-3-yl)-1-[(naphthalen-1- | | |
| | ylmethyl)-carbamoyl]-ethyl}-butyramide | | |
| 2:39 | 4-Amino-N-[1-(benzyl-phenyl-carbamoyl)-2-(1H- | | |
| | indol-3-yl)-ethyl]-butyramide | | |
| 2:40 | 4-Amino-N-[2-(1H-indol-3-yl)-1-(4-trifluoromethyl- | | |
| | phenylcarbamoyl)-ethyl]-butyramide | | |
| 2:41 | N-(1,2-Diphenyl-ethyl)-2-(2-methyl-1H-indol-3-yl)- | | |
| | acetamide | | |
| 2:42 | 1H-Indole-3-carboxylic acid (1,2-diphenyl-ethyl- | | |
| | amide | | |
| 2:43 | N-Benzhydryl-4-(1H-indol-3-yl)-butyramide | | |
| 2:44 | 1H-Indole-3-carboxylic acid benzhydryl-amide | | |
| 2:45 | N-Benzhydryl-2-(5-methoxy-2-methyl-1H-indol-3- | | |
| | yl)-acetamide | | |
| 2:46 | N-(1,2-Diphenyl-ethyl)-2-(5-methoxy-2-methyl-1H- | | |
| | indol-3-yl)-acetamide | | |
| 2:47 | N-Benzhydryl-nicotinamide | | |
| 2:48 | N-(1,2-Diphenyl-ethyl)-nicotinamide | | |
| 2:49 | 2-Chloro-N-(1,2-diphenyl-ethyl)-6-methyl- | | |
| | nicotinamide | | |
| 2:50 | 4-Amino-N-[1-(benzhydryl-carbamoyl)-2-(1H-indol- | HCl, H2O | |
| | 3-yl)-ethyl]-butyramide | | |
| 2:51 | 4-Amino-N-[1-(1,2-diphenyl-ethylcarbamoyl)-2- | HCl, H2O | |
| | (1H-indol-3-yl)-ethyl]-butyramide | | |

### Example 3

This example illustrates the potency of some of the compounds of formula (I) and their therapeutically active acid addition salts for the treatment of mental disorders.

Test 1. Affinity for the MC1-Receptor

The binding assay was carried out essentially as described by Lunec et al Melanoma Res 1992; 2; 5-12 using I¹²⁵α-NDP-MSH as ligand.

Test 2. Affinity for the MC3-Receptors, the MC4-Receptors and the MC5-Receptors

The binding assays were carried out essentially as described by Szardenings et al., J. Biol. Chem. 1997; 272; 27943-27948 and Schiöth et al., FEBS Lett. 1997; 410; 223-228 using I¹²⁵-NDP-α-MSH as ligand.

Essentially, the affinity of the compounds to the different receptors were determined using either insect cells (Sf9) or COS cells, which were transfected with recombinant human MC3, MC4 or MC5 receptors. For the determination of the affinity to the MC1 receptor, B16 mouse melanoma cells were used, which endogenously express the (mouse) MC1 receptor.

The compounds were tested at different concentrations for their ability to displace I¹²⁵-labelled NDP-MSH from the respective receptor. Incubation was performed in 96-well plates using 50,000 cells/well (Sf9 or COS cells) up to 200,000 cells/well (mouse melanoma cells).

The test compound or standard (NDP-MSH) was added in an appropriate concentration (generally between 10⁻⁴ M and 10⁻¹² M) together with labelled tracer (approx. 50,000 cpm/well) and incubation was performed for 2 hours (at room temperature for Sf 9 cells and at +37° C. for COS cells and mouse melanoma cells).

After the incubation, the cells were washed twice to get rid of excess tracer and compound, and the cells were lysed with 0.1M NaOH. The lysate was counted in a gamma-counter, binding was calculated and the affinity then determined.

Test 3. cAMP Assay p The stimulation of cAMP was carried out essentially as described by Schiöth et al., Br. J. Pharmacol. 1998; 124; 75-82.

Essentially, the effects of the compounds were tested in vivo for their ability to stimulate the production of cAMP. The cells used were the same ones that were used for the binding assays (see above), i.e. for the MC1 receptor mouse melanoma B16 cells were used and for the MC3, MC4 and MC5 receptors, Sf9 or COS cells, transfected with the respective human receptors.

Cyclic AMP was stimulated by the addition of the compounds at different concentrations in the presence of a phosphodiesterase inhibitor, during a period of 20 minutes at +37° C. cAMP was extracted with PCA, neutralised with KOH and the mixture was then centrifuged.

The concentration of cAMP was determined using a binding assay comprising binding protein (from bovine adrenals). Tritiated cAMP, used as tracer, and extracts (from above) in different dilutions were incubated at +4° C. for 120-150 minutes. The cAMP in the unknown samples displaced the labelled cAMP from binding to the binding protein. The binding protein-cAMP/tracer complex was harvested using a filter technique and the filters were counted using a beta-counter. The concentrations of cAMP in the unknown extracts were calculated using a standard curve of known concentrations.

TABLE 1
Affinity for MC-receptors

| | Ki(μM) | | | |
|---|---|---|---|---|
| Compound | MC1 | MC3 | MC4 | MC5 |
| 1:6 | 12.7 | 37.1 | 25.2 | 30.8 |
| 1:15 | 1.3 | 23.5 | 5.6 | 35.6 |
| 1:17 | 0.6 | 35.3 | 3.1 | 43.7 |
| 2:6 | 2.7 | nb | 20.8 | 17.9 |
| 2:7 | 2.6 | nb | 18.1 | 25.1 |

TABLE 1b
Influence on cAMP (given as percentage of the baseline)

| | MC1c | MC3c | MC4c | MC5c |
|---|---|---|---|---|
| 1:6 | 375 | nd | 99 | 76 |
| 2:6 | 354 | nd | 61 | 117 |
| 2:7 | 315 | nd | 79 | 154 |
| 2:14 | 162 | 116 | 237 | 164 |

### Example 4

In Vivo Effects on Food Intake

Compounds have been tested for their effects on food intake and body weight in rats.

In order to investigate the agonistic effect, i.e. decrease in food intake, of compounds, the nocturnal protocol was used. Sprague-Dawley, male rats were used, which were cannulated intracerebroventricularly. Stainless steel guide cannulae were placed in the lateral ventricle and fixed in the skull. Animals were acclimatized for a week before the experiments took place. After the experiments were done, the rats were killed and placement of the cannulae were checked.

Nocturnal Protocol:

Rats were cannulated as described above. They were used without prior starvation, and compounds were administered at 5 pm in a total volume of 5 μl. Doses of Compound 2:4 used were 1, 4 and 10 nmoles. Food intake was measured at 3, 15 and 24 hours after dosing, and body weight was recorded at 24 hours. For comparison, the well known MC4 receptor agonist, Melanotan II (MTII) was used, at a dose of 1 nmole.

Results:

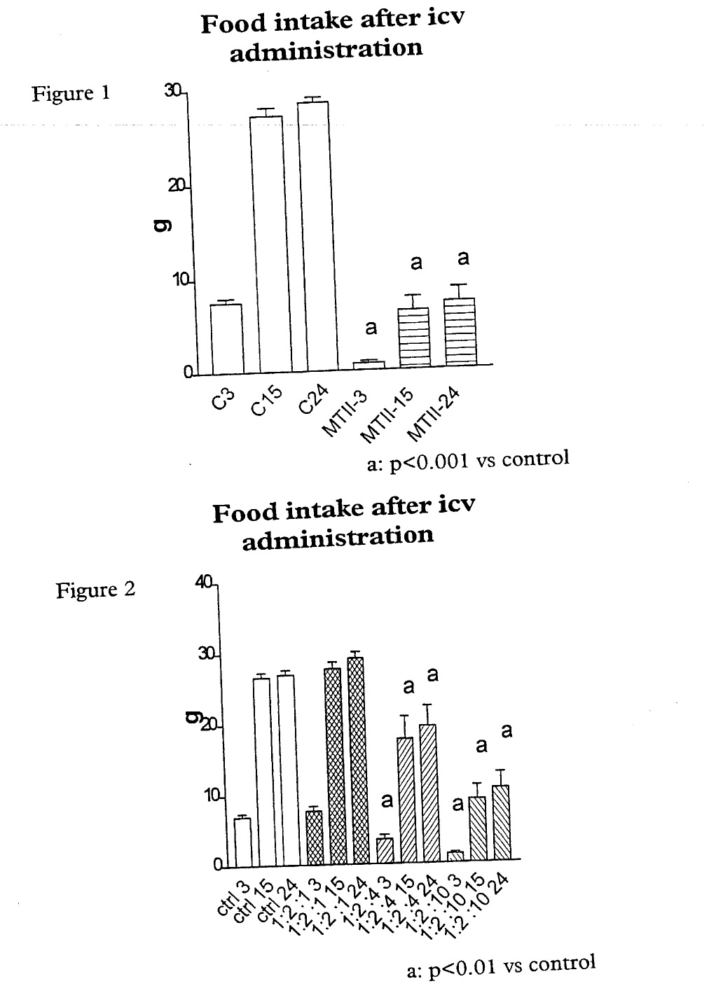

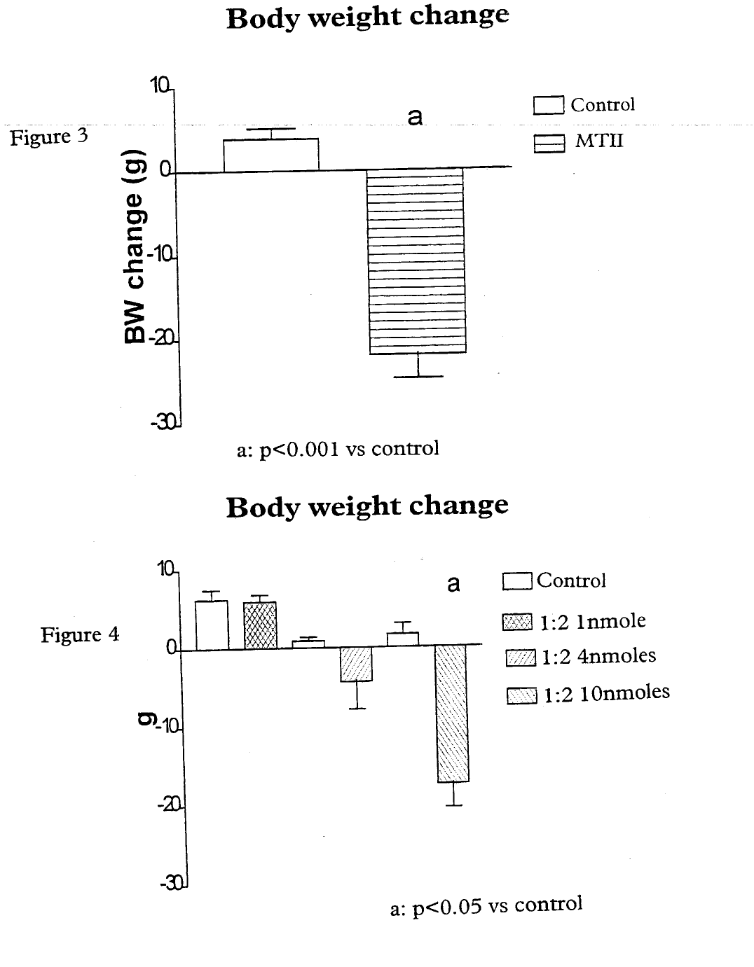

FIGS. 1 to 4 show the resulst of three different doses of Compound 2:4 given icv, and in comparison the results after the administration of MTII. There is clear dose dependency, and the intermediate and the highest dose is significantly different from vehicle treated animals regardoing food intake. The effect on body weight gain is also dose dependent and significantly different from vehicle treated animals at the highest dose tested. The effects at the highest dose was in the same range as that observed with MTII.

### Example 5

Anti-Inflammatory Effects

Control

Female BALB/c mice (weight 20-22 g) were sensitized by treatment of the shaved abdomen with 30 μl of 0.5% 2,4-dinitrofluorobenzene (DNFB). After 4 days they were challenged with 10 μl of 0.3% DNFB to the paw. The unchallenged mice paws served as a control. Twenty-four hours after the last challenge, the difference in paws weight were determined as an indicator of the inflammation (paw oedema).

Alpha-MSH and Prednisolone Controls

Mice were treated as the control but were additionally injected i.p. with α-MSH (0.5 mg/kg) or prednisolone (20 mg/kg) two hours before sensitization (day 0) and the same dose was administered repeatedly after sensitization during four consecutive days. The paw oedema inhibition was measured as described above.

Study of New Compounds

Mice were treated as the control but were additionally injected i.p. with various doses (0.05, 0.15 or 0.25, 0.375, 0.5 and 0.75 mg/kg) of each compounds two hours before sensitization (day 0) and the same dose was administered repeatedly after sensitization during four consecutive days. The paw edema inhibition as described above. Groups containing at least 10 mice each were used for all experiments.

Blood analysis was carried out using the QBC® Autoread™ Plus & QBC® Accutube System (Becton Dickinson). In all cases blood samples were collected twenty-four hours after the last challenge.

Results:

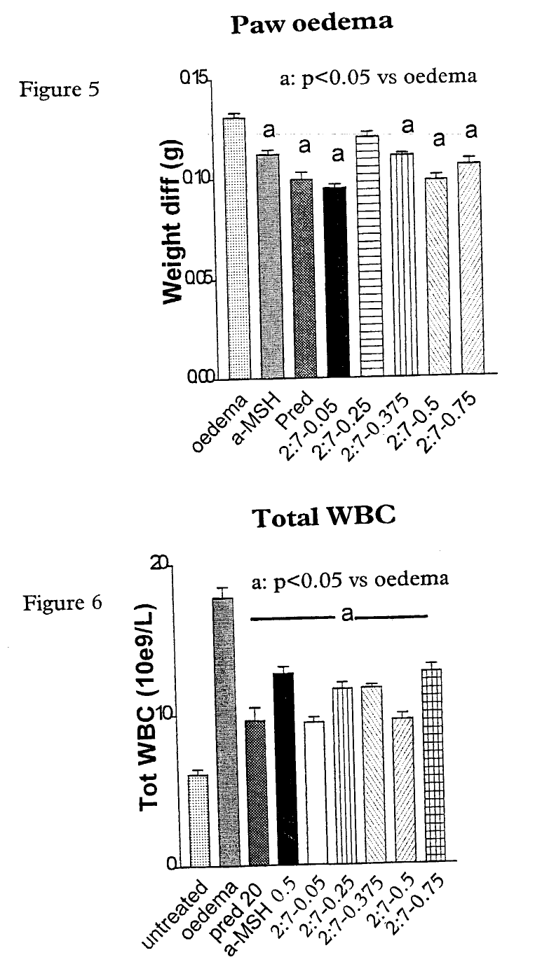

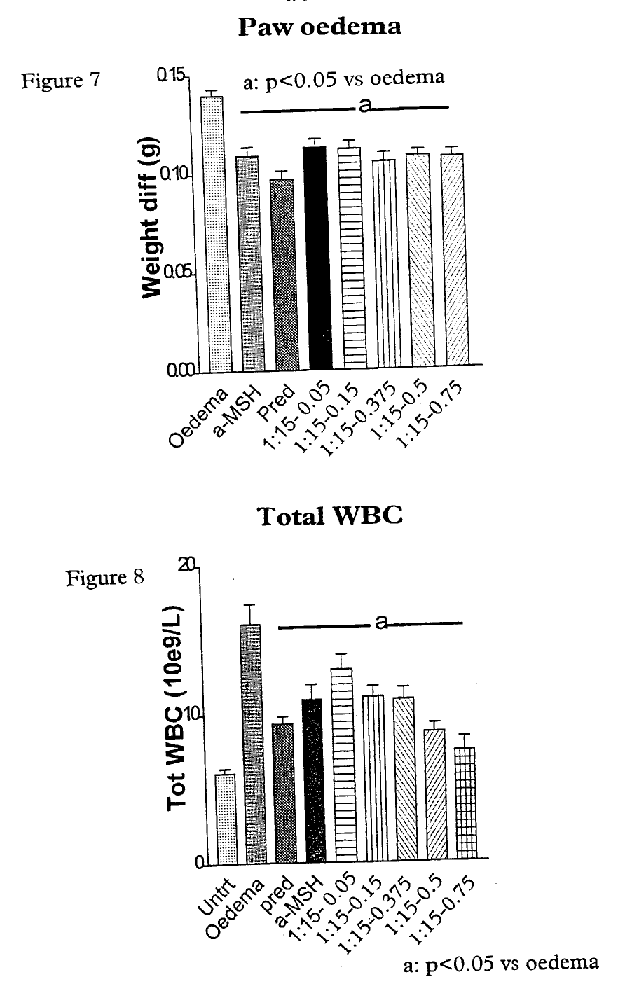

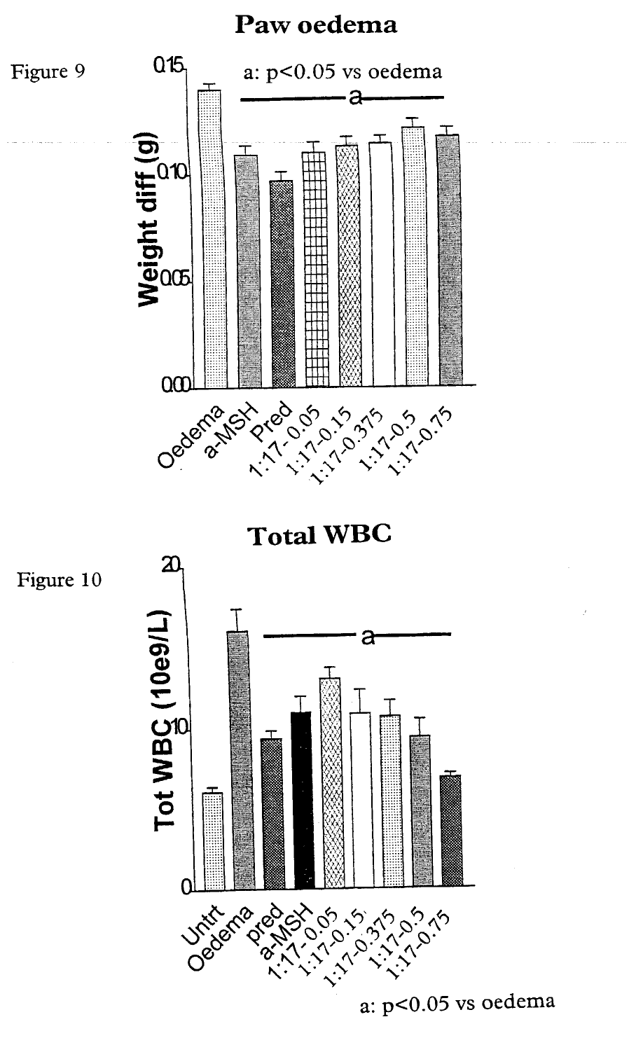

Three compounds were tested in this model. FIGS. 5-10 show the effects of these compounds on the paw oedema and total number of white blood cells. All compounds significantly decreased paw oedema compared to untreated animals (with oedema), but the most pronounced effects were observed on white blood cell count. The effects of Compounds 1:15 and 1:17 were clearly dose dependent on the white blood cell count.

The results indicate that these compounds are effective in decreasing inflammatory responses.

The following formulations are representative for all of the pharmacologically active compounds of the invention.

### Example 6

Example of a Preparation Comprising a Capsule

| | Per capsule |
|---|---|
| Active ingredient, as salt | 5 mg |
| Lactose | 250 mg |
| Starch | 120 mg |
| Magnesium stearate | 5 mg |
| Total up to | 385 mg |

In cases where higher amounts of active ingredient are required, the amount of lactose used may be reduced.

Example of a Suitable Tablet Formulation.

| | Per tablet |
|---|---|
| Active ingredient, as salt | 5 mg |
| Potato starch | 90 mg |
| Colloidal Silica | 10 mg |
| Talc | 20 mg |
| Magnesium stearate | 2 mg |
| 5% aqueous solution of gelatine | 25 mg |
| Total up to | 385 mg |

A solution for parenteral administration by injection can be prepared in an aqueous solution of a water-soluble pharmaceutically acceptable acid addition salt of the active substance preferably in a concentration of 0.1% to about 5% by weight. These solutions may also contain stabilising agents and/or buffering agents.

1. A compound of general formula (I): 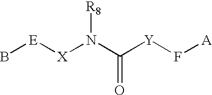
wherein E and F are independently a saturated or unsaturated, acyclic hydrocarbon group having 1, 2, 3, 4 or 5 carbon atoms;
wherein X and Y are independently methylene; one of X and Y are absent (i.e. a single bond);
or X can be: 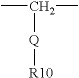
and/or Y can be: 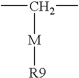
wherein M and Q are independently a saturated or unsaturated straight or branched chain acyclic hydrocarbon group having 1, 2, 3, 4, 5 or 6 carbon atoms; or M and/or Q are absent (i.e. M and/or Q are single bonds);
R8, R9 and R10 are selected independently from hydrogen and the following: 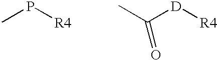
wherein P and D are independently a saturated or unsaturated, straight or branched chain acyclic hydrocarbon group having 1, 2, 3, 4, 5 or 6 carbon atoms, preferably 1, 2, 3, 4 or 5 carbon atoms; or D is absent (i.e. D is a single bond);
R4 is hydroxy, methyl, cyclohexyl, cyclopentyl, aminoguanidine, guanidine, carboxylic,
or R4 is selected from: 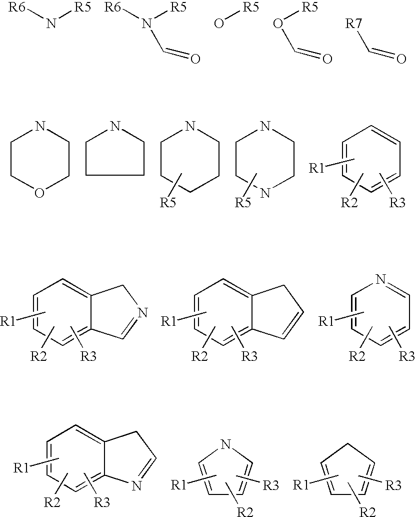
wherein R4 in R8, R9 and R10 may be the same or different.
R5 and R6 are the same or different and are selected from hydrogen, lower alkyl such as methyl, ethyl, propyl, iso-propyl, butyl, t-butyl, pentyl, t-pentyl, iso-pentyl, cyclopropyl, cyclobutyl, cyclopentyl cyclohexyl and hexyl;
R7 is selected from: 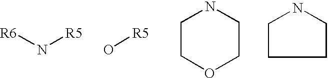
or R7 may be any one of R5 and R6;
A and B are the same or different and are selected from the following: 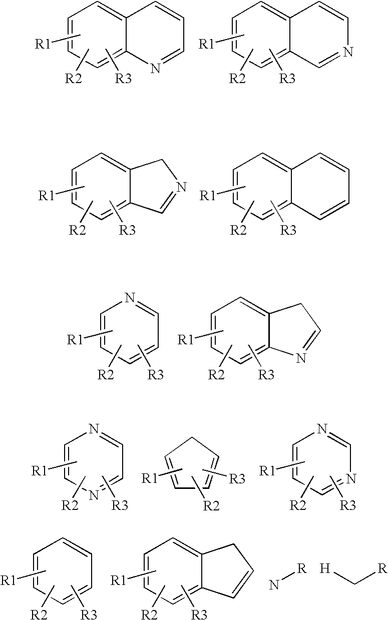
wherein R₁, R₂ and R₃ are the same or different and are selected from hydrogen, halogen, alkyl having 1 to 5 carbon atoms, electron donor groups such as alkoxy having 1-5 carbon atoms or hydroxy, electron acceptor groups selected from cyano. nitro, trifluoroalkyl or amide;
and a pharmacologically active salt thereof.

2. A compound as claimed in claim 1, wherein A and B are independently selected from:

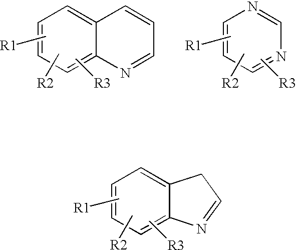

3. A compound as claimed in any one of the previous claims, wherein one or more of R1, R2 and R3 are alkyl having 1 to 5 carbon atoms.

4. A compound as claimed in claim 3, wherein the alkyl is methyl or ethyl.

5. A compound as claimed in any one of the previous claims wherein one or more of R1, R2 and R3 are alkoxy.

6. A compound as claimed in claim 5, wherein the alkoxy is methoxy.

7. A compound as claimed in any one of the previous claims wherein one or more of R1, R2 and R3 are halogen atoms.

8. A compound as claimed in claim 7 wherein the halogen is fluoro or chloro.

9. A compound having one of the following formulae:
1:2 N-Cyclohexyl-2-[[2-(1H-indol-3-yl)-ethyl]-(2-naphthalen-1-yl-acetyl)-amino]-2-naphthalen-1-yl-acetamide
1:3 1H-Indole-2-carboxylic acid [(4-cyano-phenyl)-cyclohexylcarbamoyl-methyl]-[2-(1H-indol-3-yl)-ethyl]-amide
1:4 N-Cyclohexyl-2-{(2-1H-indol-3-yl-acetyl)-[2-(1H-indol-3-yl)-ethyl]-amino}-2-pyridin-3-yl-acetamide
1:5 2-(4-Chloro-phenyl)-N-cyclohexyl-2-[[2-(1H-indol-3-yl)-ethyl]-(2-naphthalen-2-yl-acetyl)-amino]-acetamide
1:6 N-(3-Amino-propyl)-3-(1H-indol-3-yl)-2-(2-naphthalen-1-yl-acetylamino)-propionamide
1:7 N-(3-Amino-propyl)-3-(1H-indol-3-yl)-2-(2-naphthalen-2-yl-acetylamino)-propionamide
1:8 N-(3-Amino-propyl)-3-(1H-indol-3-yl)-2-(2-1H-indol-3-yl-acetylamino)-propionamide
1:9 N-(3-Guanidino-propyl)-3-(1H-indol-3-yl)-2-(2-naphthalen-2-yl-acetylamino)-propionamide
1:10 N-(3-Amino-propyl)-3-(1H-indol-3-yl)-2-(3-1H-indol-3-yl-propionylamino)-propionamide
1:11 N-[1-(3-Amino-propylcarbamoyl)-2-(1H-indol-3-yl)-ethyl]4-(1H-indol-3-yl)-butyramide
1:12 N-(3-Guanidino-propyl)-3-(1H-indol-3-yl)-2-(2-1H-indol-3-yl-acetylamino)-propionamide
1:13 N-Cyclohexyl-2-[[2-(1H-indol-3-yl)-ethyl]-(3-phenyl-propionyl)-amino]4-phenyl-butyramide
1:14 N-Benzyl-N-(4-guanidino-butyl)-3-(1H-indol-3-yl)-2-(2-1H-indol-3-yl-acetylamino)-propionamide
1:15 N-[1-[Benzyl-(4-guanidino-butyl)-carbamoyl]-2-(1H-indol-3-yl)-ethyl]-4-phenyl-butyramide
1:16 3-Benzo[1,3]dioxol-5-yl-N-[1-[benzyl-(4-guanidino-butyl)-carbamoyl]-2-(1H-indol-3-yl)-ethyl]-propionamide
1:17 N-Benzyl-N-(4-guanidino-butyl)-3-(1H-indol-3-yl)-2-(2-naphthalen-2-yl-acetylamino)-propionamide
1:18 N-[(4-Cyano-phenyl)-cyclohexylcarbamoyl-methyl]-N-(2-dimethylamino-ethyl)-3-(1H-indol-3-yl)-propionamide
1:19 N-Benzhydryl-2-(2-methyl-1H-indol-3-yl)-acetamide
1:20 N-(1,2-Diphenyl-ethyl)-4-(1H-indol-3-yl)-butyramide
1:21 N-(1,2-Diphenyl-ethyl)-2,6-dimethoxy-nicotinamide
1:22 N-Benzhydryl-2,6-dimethoxy-nicotinamide
1:23 2-(2-Bromo-phenyl)-N-cyclohexyl-2-[(2-dimethylamino-ethyl)-(2-1H-indol-3 -yl-acetyl)-amino]-acetamide
1:24 2-[[2-(5-Bromo-1H-indol-3-yl)-acetyl]-(2-dimethylamino-ethyl)-amino]-2-(2-bromo-phenyl)-N-cyclohexyl-acetamide
1:25 N-Benzhydryl-2-chloro-6-methyl-nicotinamide
2:2 4-Guanidino-N-[2-(1H-indol-3-yl)-1-phenethylcarbamoyl-ethyl]-butyramide
2:3 4-Guanidino-N-[2-(1H-indol-3-yl)-1-(3-phenyl-propylcarbamoyl)-ethyl]-butyramide
2:4 4-Guanidino-N-{2-(1H-indol-3-yl)-1-[2-(1H-indol-3-yl)-ethylcarbamoyl]-ethyl}-butyramide
2:5 4-Guanidino-N-{2-(1H-indol-3-yl)-1-[2-(1H-indol-3-yl)-ethylcarbamoyl]-ethyl}-butyramide
2:6 N-[1-(9-Ethyl-9H-carbazol-3-ylcarbamoyl)-2-(1H-indol-3-yl)-ethyl]4-guanidino-butyramide
2:7 4-Amino-N-[1-(9-ethyl-9H-carbazol-3-ylcarbamoyl)-2-(1H-indol-3-yl)-ethyl]-butyramide
2:8 4-Amino-N-[2-(1H-indol-3-yl)-1-(naphthalen-2-ylcarbamoyl)-ethyl]-butyramide
2:9 N-[2-(1H-Indol-3-yl)-1-(naphthalen-2-ylcarbamoyl)-ethyl]4-(3-methyl-thioureido)-butyramide
2:10 2-(3-Guanidino-propionylamino)-3-(1H-indol-3-yl)-N-[2-(1H-indol-3-yl)-ethyl]-propionamide
2:11 4-Amino-N-[1-(9-ethyl-9H-carbazol-3-ylcarbamoyl)-2-(5-hydroxy-1H-indol-3-yl)-ethyl]-butyramide
2:12 4-Amino-N-[2-(1H-indol-3-yl)-1-(4-phenyl-butylcarbamoyl)-ethyl]-butyramide
2:13 4-Amino-N-[2-(5-hydroxy-1H-indol-3-yl)-1-(4-phenyl-butylcarbamoyl)-ethyl]-butyramide
2:14 2-(3-Amino-propionylamino)-N-(9-ethyl-9H-carbazol-3-yl)-3-(1H-indol-3-yl)-propionamide
2:15 4-Amino-N-[2-(5-hydroxy-1H-indol-3-yl)-1-phenethylcarbamoyl-ethyl]-butyramide
2:16 4-Amino-N-[1-[2-(3,4-dimethoxy-phenyl)-ethylcarbamoyl]-2-(5-hydroxy-1H-indol-3-yl)-ethyl]-butyramide
2:17 4-Amino-N-[2-(5-methyl-1H-indol-3-yl)-1-phenethylcarbamoyl-ethyl]-butyramide
2:18 2-(3-Amino-propionylamino)-3-(1H-indol-3-yl)-N-phenethyl-propionamide
2:19 2-(3-Amino-propionylamino)-3-(1H-indol-3-yl)-N-[2-(1H-indol-3-yl)-ethyl]-propionamide
2:20 4-Amino-N-[1-benzylcarbamoyl-2-(1H-indol-3-yl)-ethyl]-butyramide
2:21 Piperidine4-carboxylic_acid_{—}[1-(9-ethyl-9H-carbazol-3-ylcarbamoyl)-2-(1H-indol-3-yl)-ethyl]-amide
2:22 Piperidine4-carboxylic_acid_{—}{2-(1H-indol-3-yl)-1-[2-(1H-indol-3-yl)-ethylcarbamoyl]-ethyl}-amide
2:23 2-(3-Amino-propionylamino)-N-[2-(3,4-dimethoxy-phenyl)-ethyl]-3-(1H-indol-3-yl)-propionamide
2:24 Piperidine-4-carboxylic_acid_{—}[2-(1H-indol-3-yl)-1-phenethylcarbamoyl-ethyl]-amide
2:25 2-(3-Amino-propionylamino)-3-(1H-indol-3-yl)-N-(3-phenyl-propyl)-propionamide
2:26 4-Amino-N-[1-[(benzo[1,3]dioxol-5-ylmethyl)-carbamoyl]-2-(1H-indol-3-yl)-ethyl]-butyramide
2:27 2-(3-Amino-propionylamino)-3-(1H-indol-3-yl)-N-(4-phenyl-butyl)-propionamide
2:28 4-Amino-N-[2-(1H-indol-3-yl)-1-(quinolin-4-ylcarbamoyl)-ethyl]-butyramide
2:29 4-Amino-N-[2-(1H-indol-3-yl)-1-(pyridin-2-ylcarbamoyl)-ethyl]-butyramide
2:30 4-Amino-N-[1-(indan-2-ylcarbamoyl)-2-(1H-indol-3-yl)-ethyl]-butyramide
2:31 4-Amino-N-[2-(1H-indol-3-yl)-1-(3,4,5-trimethoxy-benzylcarbamoyl)-ethyl]-butyramide
2:32 4-Amino-N-{2-(1H-indol-3-yl)-1-[(naphthalen-2-ylmethyl)-carbamoyl]-ethyl}-butyramide
2:33 4-Amino-N-[1-(1,2-diphenyl-ethylcarbamoyl)-2-(1H-indol-3-yl)-ethyl]-butyramide
2:34 4-Amino-N-[2-(1H-indol-3-yl)-1-(pyridin-3-ylcarbamoyl)-ethyl]-butyramide
2:35 4-Amino-N-[2-(1H-indol-3-yl)-1-(quinolin-6-ylcarbamoyl)-ethyl]-butyramide
2:36 4-Amino-N-[2-(1H-indol-2-yl)-1-(4-trifluoromethyl-phenylcarbamoyl)-ethyl]-butyramide
2:37 4-Amino-N-[1-(9-ethyl-9H-carbazol-3-ylcarbamoyl)-2-phenyl-ethyl]-butyramide
2:38 4-Amino-N-{2-(1H-indol-3-yl)-1-[(naphthalen-1-ylmethyl)-carbamoyl]-ethyl}-butyramide
2:39 4-Amino-N-[1-(benzyl-phenyl-carbamoyl)-2-(1H-indol-3-yl)-ethyl]-butyramide
2:40 4-Amino-N-[2-(1H-indol-3-yl)-1-(4-trifluoromethyl-phenylcarbamoyl)-ethyl]-butyramide
2:41 N-(1,2-Diphenyl-ethyl)-2-(2-methyl-1H-indol-3-yl)-acetamide
2:42 1H-Indole-3-carboxylic acid (1,2-diphenyl-ethyl)-amide
2:43 N-Benzhydryl4-(1H-indol-3-yl)-butyramide
2:44 1H-Indole-3-carboxylic acid benzhydryl-amide
2:45 N-Benzhydryl-2-(5-methoxy-2-methyl-1H-indol-3-yl)-acetamide
2:46 N-(1,2-Diphenyl-ethyl)-2-(5-methoxy-2-methyl-1H-indol-3-yl)-acetamide
2:47 N-Benzhydryl-nicotinamide
2:48 N-(1,2-Diphenyl-ethyl)-nicotinamide
2:49 2-Chloro-N-(1,2-diphenyl-ethyl)-6-methyl-nicotinamide
2:50 4-Amino-N-[1-(benzhydryl-carbamoyl)-2-(1H-indol-3-yl)-ethyl]-butyramide
2:51 4-Amino-N-[1-(1,2-diphenyl-ethylcarbamoyl)-2-(1H-indol-3-yl)-ethyl]-butyramide
or a pharmaceutically acceptable salt thereof.

10. A compound as claimed in any one of the previous claims which additionally comprises a label, preferably a radioactive label, or a toxic agent.

11. A pharmaceutical composition comprising a compound as claimed in any one of claims 1 to 10, together with one or more adjuvants, carriers or excipients.

12. A compound as claimed in any one of claims 1 to 10 for use as a medicament.

13. Use of a compound as claimed in any one of claims 1 to 10 in the production of a medicament for the treatment of inflammation.

14. Use of a compound as claimed in any one of claims 1 to 10 in the production of a medicament for the treatment of mental disorders.

15. Use of a compound as claimed in any one of claims 1 to 10 in the production of a medicament for the treatment of dysfunctions of the endocrine system or an hormonal system.

16. Use of a compound as claimed in any one of claims 1 to 10 in the production of a medicament for the treatment of sexual functions and/or sexual dysfunctions.

17. Use of a compound as claimed in any one of claims 1 to 10 in the production of a medicament for the treatment of drug-induced disorders of the blood and/or lymphoid system.

18. Use of a compound as claimed in any one of claims 1 to 10 in the production of a medicament for the treatment of allergic disorders.

19. Use of a compound as claimed in any one of claims 1 to 10 in the production of a medicament for the treatment of disorders of the cardiovascular system.

20. Use of a compound as claimed in any one of claims 1 to 10 in the production of a medicament for the treatment of pain.

21. Use of a compound as claimed in any one of claims 1 to 10 in the production of a medicament for inducing skin tanning or for inducing lighter skin colour.

22. Use of a compound as claimed in any one of claims 1 to 10 in the production of a medicament for the treatment of diabetes type II.

23. Use of a compound as claimed in any one of claims 1 to 10 in the production of a medicament for the treatment of obesity.

24. Use of a compound as claimed in any one of claims 1 to 10 in the production of a medicament for the treatment of anorexic conditions such as those caused by cancer, cachexia, geriatric conditions, HIV, trauma and psychological conditions.

25. Use of a compound as claimed in any one of claims 1 to 10 in the production of a medicament for inducing peripheral nerve regeneration.

26. Use of a compound as claimed in any one of claims 1 to 10 in the production of a medicament for inducing central nerve regeneration.

27. A method of treating inflammation comprising the use or administration of a compound as claimed in any one of claims 1 to 10.

28. A method of treating mental disorders comprising the use or administration of a compound as claimed in any one of claims 1 to 10.

29. A method of treating dysfunctions of the endocrine system or an hormonal system comprising the use or administration of a compound as claimed in any one of claims 1 to 10.

30. A method of treating sexual functions and/or sexual dysfunctions comprising the use or administration of a compound as claimed in any one of claims 1 to 10.

31. A method of treating drug-induced disorders of the blood and/or lymphoid system comprising the use or administration of a compound as claimed in any one of claims 1 to 10.

32. A method of treating disorders of the cardiovascular system comprising the use or administration of a compound as claimed in any one of claims 1 to 10.

33. A method of treating pain comprising the use or administration of a compound as claimed in any one of claims 1 to 10.

34. A method of inducing skin tanning or for inducing lighter skin colour comprising the use or administration of a compound as claimed in any one of claims 1 to 10.

35. A method of treating diabetes type II comprising the use or administration of a compound as claimed in any one of claims 1 to 10.

36. A method of treating obesity comprising the use or administration of a compound as claimed in any one of claims 1 to 10.

37. A method of treating anorexic conditions such as those caused by cancer, cachexia, geriatric conditions, HIV, trauma and psychological conditions comprising the use or administration of a compound as claimed in any one of claims 1 to 10.

38. A method of inducing peripheral nerve regeneration comprising the use or administration of a compound as claimed in any one of claims 1 to 10.

39. A method of inducing central nerve regeneration comprising the use or administration of a compound as claimed in any one of claims 1 to 10.

40. Use of a compound as claimed in any one of claims 1 to 10 in the production of a medicament for the treatment of skin disorders, including for the treatment of melanoma.

41. Use of a compound as claimed in any one of claims 1 to 10 in the production of a medicament for the treatment and/or diagnosis of malignancies, such as melanoma and metastases.

42. A method of treating a skin disorder, including the treatment of melanoma, comprising the use or administration of a compound as claimed in any one of claims 1 to 10.

43. A method of treating and/or diagnosing malignancies, such as melanoma and metastases, comprising the use or administration of a compound as claimed in any one of claims 1 to 10.

44. Use of a compound as claimed in any one of claims 1 to 10 in the production of a medicament for the treatment of ischemia and/or ischemia/reperfusion.

45. A method of treating ischemia and/or ischemia/reperfusion comprising the use or administration of a compound as claimed in any one of claims 1 to 10.
